(19) United States
(12) Patent Application Publication
(10) Pub. No.: US 2003/0120206 A1
(43) Pub. Date: Jun. 26, 2003

(54) Balloon cannulae

(76) Inventors: Richard G. Cartledge, Hollywood, FL (US); Leonard Y. Lee, New York, NY (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/294,366

(22) Filed: Nov. 14, 2002

Related U.S. Application Data
(60) Provisional application No. 60/344,942, filed on Dec. 21, 2001.

(51) Int. Cl. A61M 29/00

(52) U.S. Cl. 604/102.03

(57) ABSTRACT

A catheter has a balloon that overlies a trough in the catheter body such that when the balloon is deflated, the balloon does not protrude beyond the outer circumference of the catheter shaft. The wall of the catheter shaft is thicker along one side than along the remainder of the circumference of the catheter shaft to permit an inflation lumen to be disposed within the thick wall section without increasing the outer diameter of the remaining portion of the circumference.

### FIELD OF THE INVENTION

The present invention relates to the fields of cardiovascular and open heart surgery. Specifically, the present invention relates to a novel design for balloon cannulae and to methods of use for such cannulae in surgical procedures involving cardiopulmonary bypass.

### BACKGROUND OF THE INVENTION

The cardiopulmonary bypass machine is one of the most important devices in the field of cardiac surgery. It has enabled cardiac surgeons to safely perform operations for virtually the entire spectrum of acquired and congenital heart diseases. Venous blood is diverted away from the heart and to the cardiopulmonary bypass machine, where it is oxygenated. The oxygenated blood is then returned to the patient via an arterial cannula. Thus the patient's body is properly oxygenated while the heart is stopped. Although it is possible to perform some cardiac procedures without the bypass machine, use of cardiopulmonary bypass remains the cornerstone of modem cardiac surgery.

To shunt venous blood away from the heart and to the cardiopulmonary bypass machine, one or more venous cannulae are used. For many types of coronary bypass surgery, blood is typically shunted out of the body via a single venous cannula. The single venous cannula is inserted through the right atrium and has its tip disposed within the inferior vena cava. Blood enters the cannula through apertures or “fenestrations” in the portion of the cannula shaft that resides within the right atrium, as well as through fenestrations adjacent the tip residing within the inferior vena cava. The single cannula thus diverts the majority of venous return out of the body and into the bypass machine.

During procedures that require actually opening the heart so that the surgeon can access one or more of its internal chambers (i.e., for certain valve and other complex procedures), it is necessary to divert substantially all of the venous blood away from the heart to ensure that blood will not obscure the surgical field. It is also preferable not to have a venous cannula traversing the right atrium, since this not only can obscure the surgeon's view of the internal structures of the heart but also can make manipulation of the heart more difficult. Additionally, it is not desirable to have the fenestrations of the venous cannulae exposed to the ambient air since the bypass circuit should remain filled only with fluid to function efficiently. For these reasons, when surgeons are to perform a procedure that requires total venous diversion, two venous cannulae are used; one to divert blood from the superior vena cava, and one to divert blood from the inferior vena cava. This “bicaval” cannulation shunts substantially all of the venous blood out of the vena cavae to a cardiopulmonary bypass machine, which oxygenates the blood and returns it to the patient via an aortic cannula.

To achieve bicaval cannulation, the surgeon first places a stitch in a circular or “purse-string” configuration at the points of insertion of each cannula, and an incision is made in the tissue central to the respective purse strings. The forward end of one venous cannula is inserted through an insertion point in the anterior wall of the superior vena cava and advanced into the superior vena cava. The second venous cannula is inserted through an insertion point in the inferior lateral aspect of the right atrium and into the inferior vena cava. After the cannulae are placed, the purse strings are cinched around them to create a seal, thus preventing external bleeding or the ingress of air into the cardiopulmonary bypass circuit.

To achieve total venous diversion, clamps or snares must be placed around the superior and inferior vena cavae and cinched down around the distal ends of the cannulae so that no venous blood can pass around and enter the heart. The act of placing these tourniquets involves posterior lateral and posterior medial dissection of the cavae. This surgical maneuver is extremely dangerous and can cause tears in the vena cavae, typically in a posterior location. Additionally, the azygous vein, which joins the superior vena cava in a posterior location, is at risk for injury during dissection of the superior vena cava and snare placement. These tears are extremely difficult to repair because of the limited visual access to their location. Since the cavae are very thin and friable, they are easily injured, with the potential consequence of massive hemorrhage with hemodynamic instability requiring massive transfusions. Such injuries usually necessitate the surgeon to “crash on bypass” before the patient is physiologically ready. If repair can be performed, the repair itself may result in significant narrowing, potentially causing significant morbidity and mortality to the patient. There is also a significant risk of death from such an injury.

Another type of cannula used in a cardiovascular bypass procedure is the arterial cannula. The arterial cannula returns oxygenated blood from the cardiopulmonary bypass machine back to the patient. One such arterial cannula is the arterial balloon cannula, which includes an elongated tubular portion and an expandable balloon at its distal end. A main lumen extends the length of the shaft and has apertures at both its proximal and distal ends. The cannula is inserted through the aortic wall and advanced until the balloon resides within the aortic lumen. When the balloon is inflated, a seal is created between the cannula shaft and the lumen of the aorta to isolate the heart from the cardiopulmonary bypass circuit, thus allowing the surgeon to operate on a non-beating, relatively bloodless organ. With alternate embodiments of arterial cannulae without a balloon, the aorta must be cross-clamped to isolate the heart from the systemic circulation.

When the procedure calls for stopping the heart, cardioplegia solution is administered into the coronary arteries. Cardioplegia solution may be administered either antegrade (thuough the aortic root and into the coronary arteries) or retrograde (in the reverse direction, from the coronary sinus into the coronary arteries). To administer the cardioplegia solution in retrograde fashion, unidirectional flow must be assured.

Stopping the flow of blood from backing out the administration site can be accomplished by a purse-string placed around the opening to the coronary sinus, with the cardioplegia solution being administered through the retrograde cannula lumen distal to the portion of the cannula shaft that is sealed by the purse-string. But most modern retrograde cardioplegia cannulae avoid the need to place a purse-string in the coronary sinus by providing a balloon affixed to the cannula that is inflated to prevent blood from flowing around the cannula in the wrong direction. Using a balloon retrograde cardioplegia cannula eliminates the time and extra steps it takes to access the coronary sinus and place the purse-string suture.

A conventional balloon retrograde cannula includes an elongated, flexible tubular shaft and an expandable balloon at its distal end. A main lumen extends the length of the shaft and is open at opposite ends of the shaft. The cannula is inserted through the right atrium and advanced until the balloon resides within the coronary sinus. When the balloon at the tip of the cannula is inflated, a seal is created between the cannula shaft and the coronary sinus wall. This seal creates an exclusive communication between the lumen of the cannula and the lumen of the coronary sinus, thus maximizing the efficiency of delivery of cardioplegic solution, blood or other agents into the heart.

Regardless of whether the cannula is a venous cannula, an arterial cannula, or a retrograde cardioplegia cannula, problems are presented with conventional balloon cannulae. The balloon of prior art cannulae is placed on the outside surface of the distal tubular portion, thus creating an acute increase in diameter where the balloon overlies the tubular portion of the cannula. Thus there is a ridge or “step up” where the edge of the balloon is attached to the cannula shaft. This step up can make placement of a conventional balloon cannula problematic, in that it presents a rough location on the surface of the cannula that can cause trauma to the walls of the vessel as the cannula is inserted and withdrawn. In the case of the aortic balloon cannulae, the step up can scrape plaque off the wall of the aorta, which then can embolize in the bloodstream, with adverse clinical consequences. Further, in addition to the main lumen, balloon cannulae must accommodate other accessory lumens for purposes such as inflation of the balloon, cardioplegia infusion pressure monitoring, etc. These conduits typically lie on the exterior surface of the tubular member of the cannula, creating an irregular, non-circular outer cross-sectional profile that can compromise the seal between the vessel/atrial wall and the tubular portion of the cannula. A compromised seal, in turn, can cause blood leakage outside of the anatomic structure the cannula is residing in and potential ingress of air into the circulatory system and/or into the cardiopulmonary bypass circuit, with undesirable clinical consequences. These and other cannulae that include accessory lumens that lie on the internal surface of the cannulae reduce the effective cross-sectional area of the principle lumen and therefore increase resistance and compromise flow of whatever fluid is flowing through the principle lumen.

### SUMMARY OF THE INVENTION

The present invention is directed towards a novel design for balloon cannulae to be used when bi-caval cannulation of the heart is indicated, eliminating the need to perform circumferential caval dissection and further reducing the tissue trauma caused by prior art balloon cannulae. Balloon cannulae according to a disclosed embodiment of the present invention comprise inflatable, occlusive balloons adjacent their distal ends. While these cannulae are inserted and positioned by a surgeon in the standard fashion, the need for circumferential dissection of the cavae and tourniquet placement is obviated. After the cannulae are positioned and secured with purse string sutures, the surgeon inflates the occlusive balloons by infusing an inflation medium with a syringe or other means. Once the balloons are inflated, all of the venous return is diverted. The balloons inflate around the distal ends of the cannulae and allow blood to flow through the lumen of the cannulae, but not around the balloons. Use of these cannulae minimizes the chance of caval injury by eliminating the need for circumferential dissection. Additionally, the configuration of the balloon in relation to the cannula is such that the balloon is “flush” with the cannula so that there exists no acute change in diameter along the external surface of the cannula, which serves to avoid tissue trauma during insertion and withdrawal into and out of bodily structures.

The present invention addresses the two major problems presented by existing designs for balloon cannulae. In various embodiments according to the present invention, the lumens are configured such that a balloon cannula can be inflated without compromising either the flow within the principle lumen of the cannula or the seal between the cannula and the structure within which the cannula lies. Moreover, a disclosed example of a cannula according to the present invention is provided with a trough within the cannula body at its distal end in which the balloon member lies such that when uninflated during insertion and withdrawal, there is a smooth interface between the external cannula wall and the uninflated balloon allowing for smoother, easier, and safer insertion and withdrawal.

### BRIEF DESCRIPTION OF THE DRAWINGS

FIG. 1 is a side perspective view of a balloon cannula of a disclosed embodiment of the present invention.

FIG. 2 is a side view of the distal portion of the balloon cannula of FIG. 1 with the balloon in an uninflated condition.

FIG. 3 is a side view of the distal portion of the balloon cannula of FIG. 1 with the balloon in an inflated condition.

FIG. 4 is a side view of the distal portion of the cannula shaft of the balloon cannula of FIG. 1 with balloon removed to show detail.

FIG. 5 is a longitudinal sectional view of the distal portion of the shaft of FIG. 4.

FIG. 6 is a cross-sectional view taken along line 6-6 of FIG. 4.

FIG. 7 is a perspective sectional view taken along line 7-7 of FIG. 4.

FIG. 8 is a longitudinal sectional view of the distal portion of the balloon cannula of FIG. 1 with the balloon in its uninflated condition.

FIG. 9 is a longitudinal sectional view of the distal portion of the balloon cannula of FIG. 1 with the balloon in its inflated condition.

FIG. 10 is a perspective view showing two balloon cannulae of the type illustrated in FIGS. 1-9 positioned within the heart of a patient for performing a cardiac bypass procedure.

FIG. 11 is a side view of an arterial balloon cannula.

### DETAILED DESCRIPTION OF THE DISCLOSED EMBODIMENT

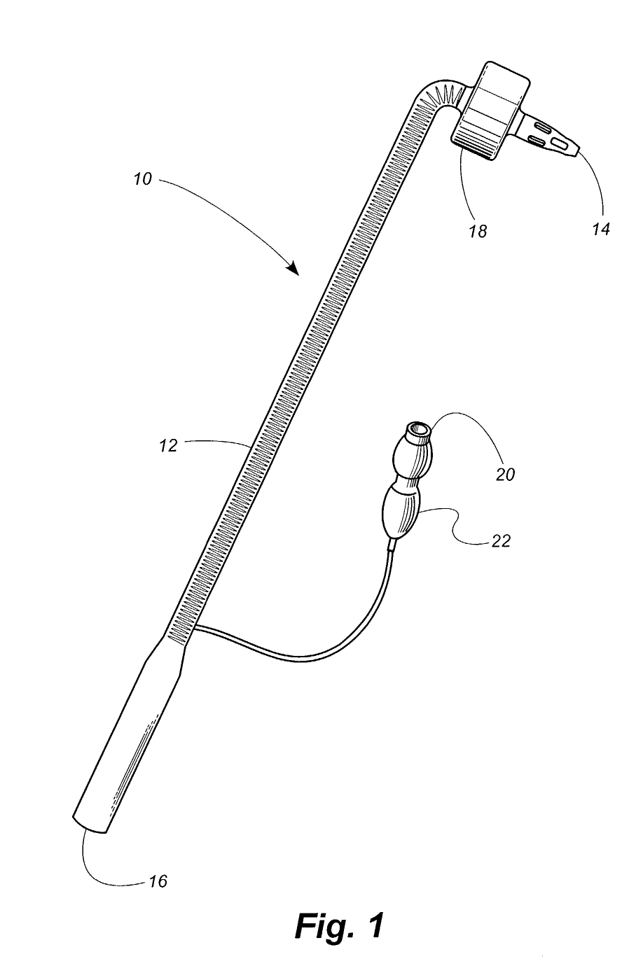

Referring now in more detail to the drawings, in which like numerals indicate like elements throughout the several views, FIG. 1 illustrates a balloon cannula 10 according to a disclosed embodiment of the present invention. The balloon cannula 10 includes an elongated, tubular cannula shaft 12 having a distal end 14 and a proximal end 16. In the disclosed embodiment, the cannula body 12 is constructed of a pliable material such as, but not limited to, natural or synthetic rubbers, elastomers, polyisoprenes, polyurethanes, vinyl plastisols, acrylic polyesters, polyvinylpyrrolidone-polyurethane interpolymers, butadiene rubbers, styrene-butadiene rubbers, rubber lattices, and other polymers or materials with similar resilience and pliability qualities. An inflatable member or balloon 18 is located adjacent the distal end 14 of the cannula shaft 12. An inflation valve 20 adapted to accommodate a syringe or other suitable apparatus branches off from the cannula shaft 12 at a location adjacent the proximal end 16 of the cannula shaft. A syringe can be coupled to the inflation valve 20 to inflate or to deflate the balloon 18 by infusing liquids or gases into the inflatable member through an inflation lumen. In the disclosed embodiment, a pilot balloon 22 is provided in-line between the inflation valve 20 and the balloon 18. The pilot balloon 22 is inflatable and is designed to provide a user an indication of the degree of inflation of the balloon 18.

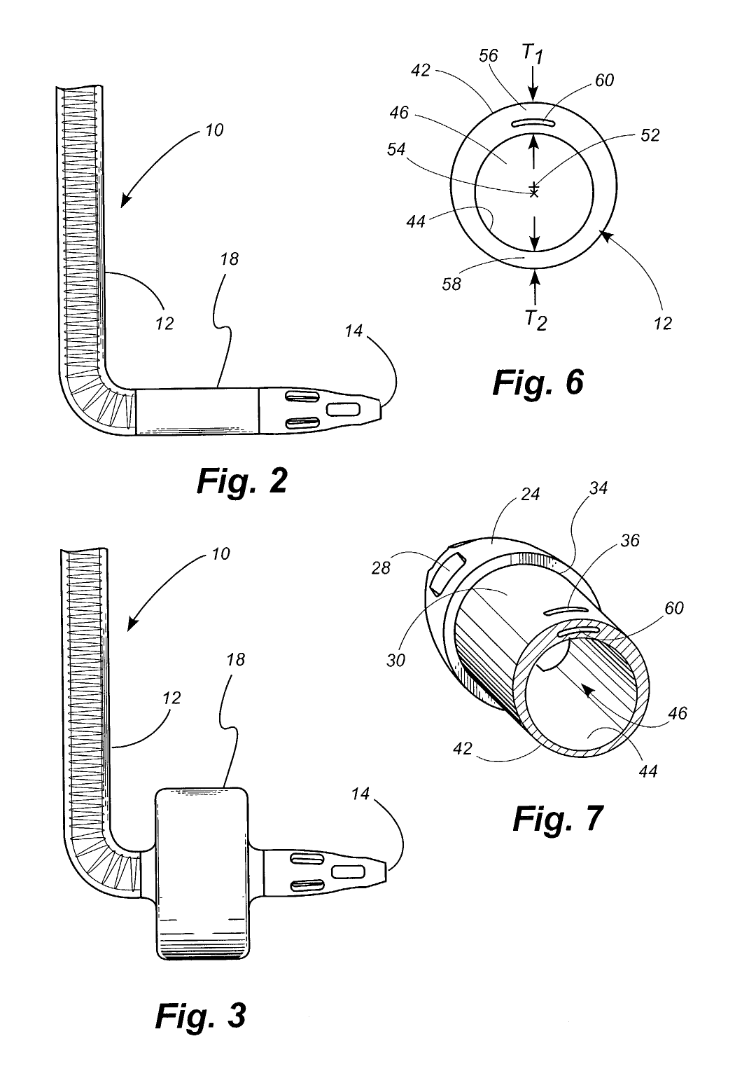

FIG. 2 illustrates the distal portion of the balloon cannula 10 with the balloon 18 in its uninflated condition. FIG. 3 illustrates the distal portion of the balloon cannula 10 with the balloon 18 in its inflated condition.

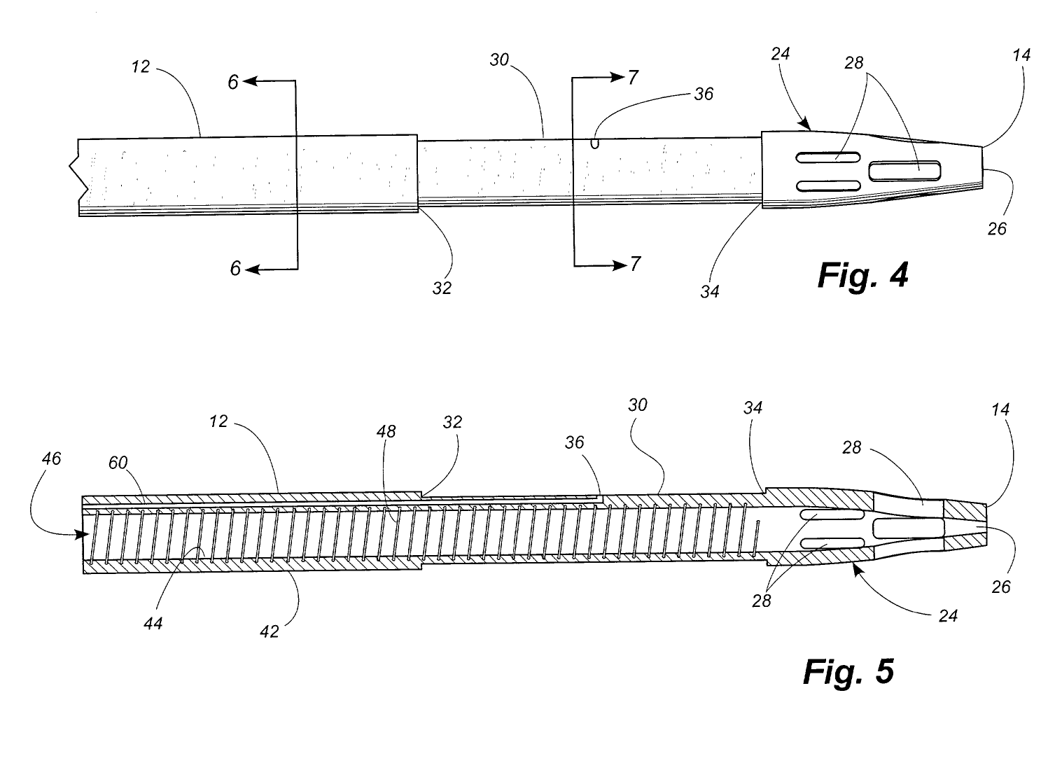

Reference is now made to FIG. 4 of the drawings, in which the distal portion of the cannula shaft 12 is illustrated. A cannula tip 24 located at the forward end of the shaft 12 is tapered to facilitate introduction. An opening 26 is formed in the forward end of the cannula tip 24. In addition, lateral openings 28 are formed in the sides of the cannula tip 24. In alternate embodiments, the cannula tip can be rounded instead of tapered, the end of the distal tip 14 may be closed instead of open, or it may be provided with one or more central apertures 26. The lateral apertures 28 may be provided in addition to or in place of the central aperture 26 or may be eliminated entirely.

Just rearward of the cannula tip 24, a portion of the shaft 12 has a reduced diameter so as to form a circumferential trough 30. The trough 30 has a proximal edge 32 and a distal edge 34. An inflation aperture 36 is formed in the wall of the trough portion 30.

Referring now to FIG. 5, the cannula shaft 12 has an exterior surface 42 and an interior surface 44. The cannula shaft 12 defines a main lumen 46 that extends the length of the shaft. The axial tip opening 26 and the lateral openings 28 place the cannula lumen 46 in fluid communication with the ambient surrounding the cannula tip 24.

To provide structural reinforcement for the main lumen 46, a coil 48 may extend along some or all of the length of the cannula shaft 12. The coil 48 is preferably embedded within the wall of the cannula shaft 12 but in alternate embodiments may wrap around the exterior of the cannula wall 18 or may be positioned within the cannula lumen 46. The coil 48 may be constructed of a wire of stainless steel or other suitable metal or of plastics sufficiently stiff to increase cannula strength and to prevent kinking of the cannula during use.

As can be seen in FIG. 6, the cannula shaft 12 has a longitudinal axis 52, and the main lumen 46 of the cannula shaft has a longitudinal axis 54. The longitudinal axis 54 of the main lumen 46 is offset from the longitudinal axis 52 of the cannula shaft 12 such that the main lumen is eccentric with respect to the cannula shaft. As a consequence, a portion 56 of the wall on one side of the main lumen 46 has a thickness T₁ that is greater than the thickness T₂ of the wall portion 58 on the opposite side of the main lumen. As shown in FIG. 7, an inflation lumen 60 is formed in the thicker portion 56 of the wall and communicates with the inflation aperture 36 in the trough portion 30 of the cannula shaft 12. The asymmetrical configuration permits the inflation lumen to be incorporated into the wall of the cannula shaft 12 to eliminate the protrusion of a separate inflation tube on the periphery of the cannula body. At the same time, since only the portion 56 of the wall defining the inflation lumen 60 is thickened, the overall circumference of the cannula shaft 12 is minimized.

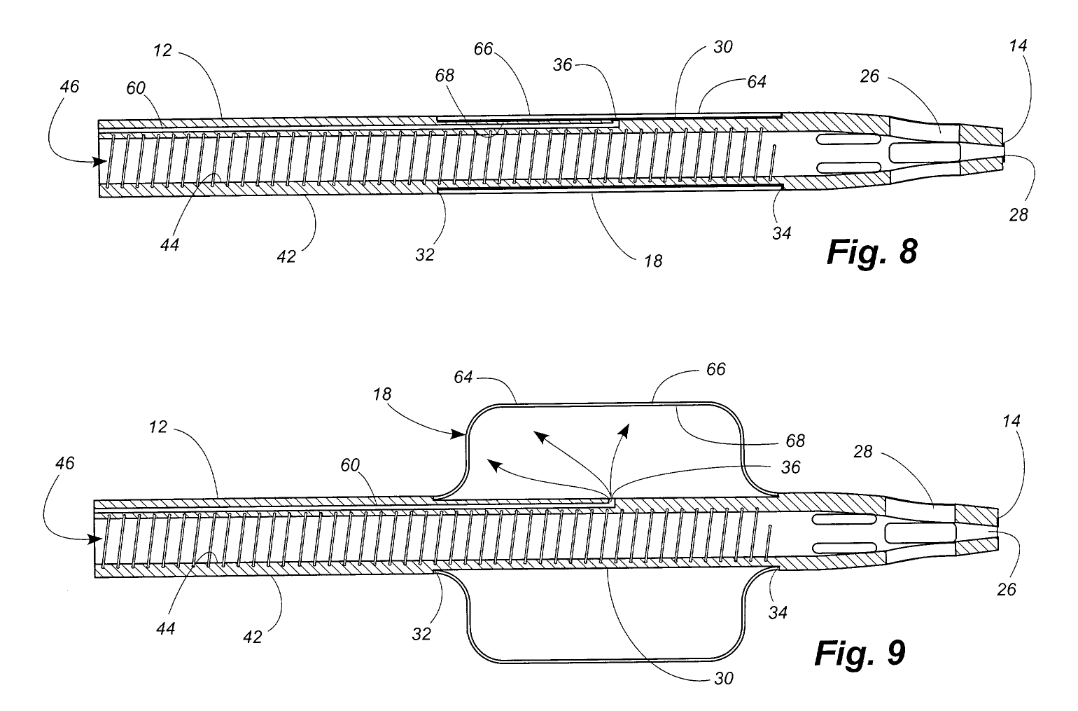

Referring now to FIG. 8, the balloon 18 is shown fastened to the cannula shaft 12 and in its uninflated condition. The balloon 18 includes a wall 64 of a fluid-impermeable material. The wall 64 of the balloon 18 has an exterior surface 66 and an interior surface 68. The balloon 18 overlies the trough portion 30 of the cannula shaft 12 and the inflation aperture 36 located thereon. The balloon 18 is secured at its rearward and forward ends to the proximal and distal end portions 32, 34 of the trough 30. In its uninflated condition, the interior surface 68 of the wall 64 of the balloon 18 is imposed against the trough portion 30 of the cannula shaft 12, and the exterior surface of the balloon is flush with the exterior surface 42 of the main portion of the cannula shaft 12. Stated differently, the reduction in diameter of the trough portion 30 is equal to the thickness of the wall 64 of the balloon 18 in its uninflated state. By recessing the balloon 18 into the trough 30 in this manner, there is no portion of enlarged circumference that might cause trauma to a bodily structure upon insertion and again upon withdrawal of the cannula 10.

FIG. 9 depicts the balloon 18 in its inflated state. An infusion medium such as a saline solution is infused by a syringe or other suitable means into the inflation port 20 (FIG. 1), through the inflation lumen 60, and out the inflation aperture 36 beneath the surface of the balloon. In response, the balloon 18 expands radially outward.

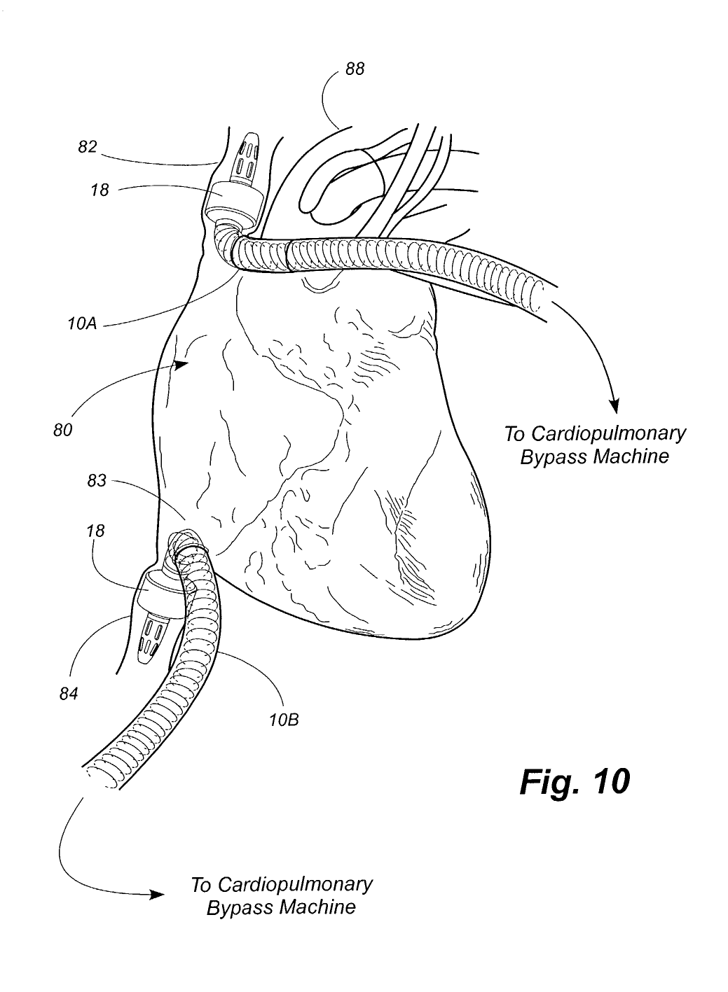

FIG. 10 illustrates the use of a pair of balloon cannulae 10 to perform a bicaval cannulation for the purpose of diverting venous blood away from a heart 80 and to a cardiopulmonary bypass machine. First, a stitch is placed in a circular or “purse-string” configuration at a first insertion point on the anterior wall of the superior vena cava 82. Another purse-string stitch is placed at a second insertion point in the inferior lateral aspect of the right atrium 83. An incision is made in the tissue central to the respective purse strings. A first balloon cannula 10A is then advanced through the circular stitch at the first insertion point and into the superior vena cava 82. A second balloon cannula 10B is advanced through the circular stitch at the second insertion point and into the inferior vena cava 84. The purse strings are then cinched around the balloon cannulae 10A, 10B to create a seal, thus preventing external bleeding or the ingress of air into the cardiopulmonary bypass circuit.

At this point sufficient blood is shunted to the cardiopulmonary bypass machine for a bypass procedure to begin. Return flow of oxygenated blood from the bypass machine is directed through an arterial cannula (not identified in FIG. 10) to the aorta 88. If it is necessary to divert the complete flow of blood, the balloons 18 of each of the cannulae 10A, 10B are inflated, forming a fluid-tight seal between the cannula shaft and the wall of the respective vena cavae 82, 84. Now all of the venous blood flow is diverted to the bypass machine, creating a clear field of view for the surgeon to operate.

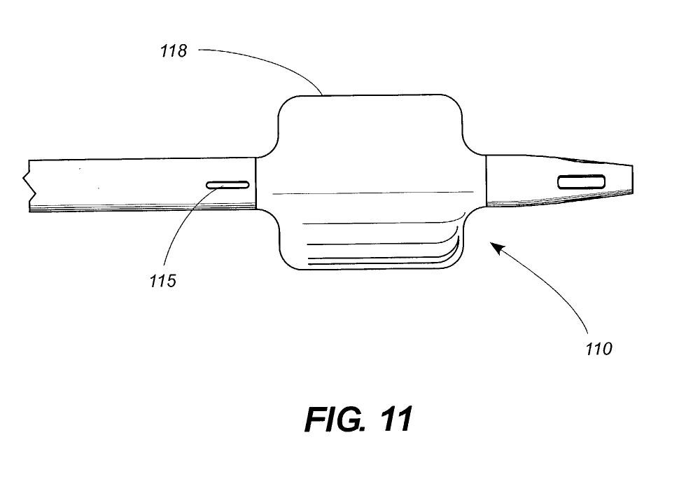

FIG. 11 illustrates a cannula 110 that is suitable for use as an arterial balloon cannula. In addition to a main lumen and an inflation lumen, the cannula 110 includes an accessory lumen that terminates in a port 115 proximal to the balloon 118. The cannula 110 is inserted through the aortic wall and advanced until the balloon resides within the aortic lumen. When the balloon 118 is inflated, a seal is created between the cannula shaft and the lumen of the aorta to isolate the heart from the cardiopulmonary bypass circuit, thus allowing the surgeon to operate on a non-beating, relatively bloodless organ. Cardioplegia can then be administered through the accessory lumen and out of the port 115.

In various embodiments according to the present invention, the thickness of the cannula wall may increase through some or all of its length, such that there may be a cannula taper in which the external diameter of the cannula shaft 12 increases towards the proximal end 16 of the cannula 10. In all cases, however, the diameter of the cannula lumen 46 as defined by the circumference of the internal cannula wall 20 remains constant throughout the length of the device 10. The constant diameter of the cannula lumen 46 serves to maintain an even flow therethrough.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the description and examples presented herein should not be construed to limit the scope of the present invention, the essential features of which are set forth in the appended claims.

What is claimed is:

1. A balloon cannula, comprising:
an elongated shaft having an exterior surface and having proximal and distal ends;
a main lumen extending substantially the entire length of said shaft;
a circumferential trough formed in said exterior surface of said elongated shaft, said circumferential trough having a reduced diameter in comparison to the adjacent circumference of said elongated shaft, and said trough having proximal and distal edges;
a balloon overlying said circumferential trough of said elongated shaft, said balloon having a wall with interior and exterior surfaces, and said wall having proximal and distal portions, said interior surface of said balloon wall being secured at said proximal and distal portions to said proximal and distal edges of said trough in a fluid-tight manner; and
an inflation lumen formed in said elongated shaft and terminating at a port opening beneath said balloon, whereby fluid can be infused and withdrawn through said inflation lumen to inflate and to deflate said balloon.

2. The balloon cannula of claim 1, wherein said balloon and said trough are configured such that when said interior surface of said balloon wall is imposed against said trough in an uninflated state, said exterior surface of said balloon wall is flush with said adjacent circumference of said elongated shaft.

3. The balloon cannula of claim 1, further comprising an accessory lumen formed within said shaft and terminating in an aperture located distal to said balloon.

4. The balloon cannula of claim 1, further comprising an accessory lumen formed within said shaft and terminating in an aperture located proximal to said balloon.

5. A balloon cannula, comprising:
an elongated shaft, said elongated shaft defining a longitudinal lumen, said elongated shaft having a central longitudinal axis, and said longitudinal lumen having a central longitudinal axis laterally offset from said central longitudinal axis of said elongated shaft;
whereby said longitudinal lumen is eccentric with respect to said elongated shaft.

6. The balloon cannula of claim 5, wherein said elongated shaft and said longitudinal lumen each have a circular cross-section.

7. The balloon cannula of claim 5,
wherein said longitudinal lumen comprises a main longitudinal lumen,
wherein said longitudinal lumen being eccentric with respect to said elongated shaft defines a wall section on a first side of said longitudinal lumen having a greater thickness than a wall section on an opposite second side of said longitudinal lumen; and
wherein said balloon cannula further comprises a second longitudinal lumen formed in said wall section of greater thickness.

8. The balloon cannula of claim 5, further comprising an accessory lumen formed within said shaft and terminating in an aperture located distal to said balloon.

9. The balloon cannula of claim 5, further comprising an accessory lumen formed within said shaft and terminating in an aperture located proximal to said balloon.
